(19) United States
(12) Patent Application Publication
(10) Pub. No.: US 2011/0143305 A1
(43) Pub. Date: Jun. 16, 2011

(54) MEDICAL OR DENTAL TREATMENT DEVICE FOR DISPENSING A MEDIUM

(75) Inventors: Hannes Wagner, Salzburg (AT); Walter Schuh, Buermoos (AT)

(73) Assignee: W&H Dentalwerk Burmoos GmbH

(21) Appl. No.: 12/955,879

(22) Filed: Nov. 29, 2010

(30) Foreign Application Priority Data
Dec. 10, 2009 (EP) 09178643.4

(51) Int. Cl. A61C 5/02; A61C 5/04; A61N 5/00; A61C 19/00

(52) U.S. Cl. 433/29; 433/86; 433/32; 433/81; 433/226

(57) ABSTRACT

A medical or dental treatment device for dispensing a medium having a base part with a power supply unit and an electrical or electronic control device and at least one delivery part that can be connected detachably to the base part via a coupling device and which has a sound source, such as an ultrasound source, and a delivery device for dispensing the medium from the delivery part. In addition, at least one active part, which can be connected detachably to the base part via the coupling device, may also be provided.

### CROSS REFERENCE TO RELATED APPLICATION

The present application claims priority from pending European Patent Application No. 09178643.4 filed Dec. 10, 2009, which is incorporated herein by reference.

### BACKGROUND

1. Field

The present invention relates to a medical or dental treatment device for dispensing a medium.

2. Description of Prior Art

Such a treatment device is known from U.S. Pat. No. 7,014,462 B1. The disadvantage of this treatment device is that during operation, the treatment device offers the user very little or no flexibility with respect to different applications or different media to be dispensed.

It would be advantageous to create a medical or dental treatment device for dispensing a medium, which offers the user more opportunities for selecting operating parameters for specific applications or for configuring the treatment device for media having different properties.

### SUMMARY

According to one embodiment, the medical or dental treatment device for dispensing a medium has a base part with a power supply unit and an electrical or electronic control device and at least one delivery part that can be connected detachably to the base part via a coupling device and comprises a sound source, in particular an ultrasound source, and a delivery device for dispensing the medium from the delivery part. According to a preferred embodiment, the treatment device has multiple delivery parts comprising in particular different sound sources. The different sound sources generate different vibrational amplitudes or vibration frequencies or they output different powers, so that the user can connect the delivery part having the suitable sound source to the base part, depending on the application or the medium to be dispensed.

According to an alternative embodiment, the treatment device has at least one active part which can be connected detachably to the base part via the coupling device and can be connected instead of the delivery part to the base part. The at least one active part (in contrast with the delivery part) does not have a delivery device for dispensing a medium. Preferably at least one delivery part and at least one active part are used together in one application but in chronological succession. According to an especially preferred embodiment, the active part is used for acting on the medium dispensed by the delivery part. “Acting on the medium” is understood in particular to refer to a chemical, physical, thermal, mechanical or electromagnetic action, processing or activation.

According to one embodiment, the treatment device is designed as a wireless (i.e., cordless) handpiece, and the power supply unit is designed as a battery or an accumulator. Alternatively, the treatment device can comprise a hardwired handpiece, a power supply line and a control unit and/or supply unit, which can be connected to the handpiece via the power supply line.

According to another embodiment, the base part has a handle section, in particular a pistol-shaped handle section, which holds at least a portion of the power supply unit and/or the electrical or electronic control device. Alternatively, the treatment device is designed as a straight or contra-angle handpiece having a base part and at least one delivery part, as described above.

The sound source, in particular the ultrasound source, is designed as a pneumatic sound source, a piezoelectric sound source or a magnetostrictive sound source, for example.

The delivery device has, for example, a movable or displaceable piston and an operating element for selective movement of the piston. The operating element is designed as a mechanical operating element in particular, for example, as a movable or pivotable lever or as an electrically operable operating element for example, as a button.

The media that can be dispensed and delivered by the treatment device comprise, for example, a filling material for dispensing into a cavity, in particular a dental cavity, a filling material for dispensing into a dental root, a rinsing agent or disinfecting agent for dispensing into a dental root or an anesthetic for intraosseous dispensing, in particular into a jawbone.

According to one embodiment, the at least one delivery part has a receptacle for the medium or a connection for a media container and a sonotrode for transferring the vibrations generated by the sound source, in particular the ultrasound source, to the medium, the receptacle and/or the connection. Thus, especially good deliverability, distribution of the medium in the target region (or activation of the medium) is achieved in an advantageous manner.

According to one embodiment, the at least one delivery part has a radiation source for dispensing a radiation which activates the medium. According to one other embodiment, the at least one delivery part has a tool for the medium, in particular a modeling probe, an intraosseous needle or a root canal probe and a sonotrode for transferring the vibrations generated by the sound source, in particular the ultrasound source, to the tool. According to another embodiment, the at least one delivery part is provided with a heat source for heating the medium. These embodiments increase the functionality of the treatment device and the user friendliness because the user can perform multiple treatment steps by using one delivery part.

As already described above, the treatment device according to one embodiment has at least one active part, which can be connected detachably to the base part via the coupling device and can be connected instead of the delivery part to the base part. The advantage of this embodiment is that due to the use of multiple different active parts in particular, the functionality of the treatment device is further increased. For example, multiple active parts having different radiation sources may be made available, wherein each radiation source emits a different radiation, for example, each having a specific wavelength and/or power. Alternatively, dental filling material dispensed onto teeth may be modeled and/or compressed, depending on requirements, by means of multiple active parts each of which has a modeling probe with an individually shaped modeling section, for example.

According to one embodiment, the at least one active part is designed to act on the medium dispensed by the delivery part. The at least one active part therefore comprises, for example, a radiation source for dispensing a radiation activating the medium and/or a tool for the medium dispensed, in particular a modeling probe or an obturation needle and/or a sound source, in particular an ultrasound source for transferring vibrations to the tool and/or a heat source for heating the medium.

According to an alternative embodiment, the at least one active part is designed to act on the treatment site, onto which the delivery part has dispensed or will dispense the medium. The at least one active part therefore comprises, for example, a tool for preparation of the root canal and preferably a sound source, in particular an ultrasound source for transferring vibrations to the tool.

The medical or dental treatment device may be used for various different applications, for example:
for dispensing filling material into a cavity, in particular a dental cavity;
for acting on the filling material dispensed by the delivery part into the cavity, in particular for irradiating the filling material and/or for compacting and/or modeling the filling materials;

wherein preferably in both applications cited above, a filling material in which a change in viscosity is achieved by acting upon the filling material with sound or ultrasound and/or heat is used, for example, a dental cement or a filling compound based on a synthetic resin, preferably a filling compound based on a synthetic resin, which can be polymerized or cured by light, in particular a highly viscous dental filling compound based on a synthetic resin and having a high inorganic filler content;
for dispensing filling material into a dental root;
for acting on the filling material dispensed by the delivery part into the dental root, in particular for heating and/or compressing and/or modeling the filling material;

wherein preferably in the two latter applications, a filling material in which a change in viscosity is achieved by exposing the filling material to sound or ultrasound and/or heat is used, for example, a dental cement or gutta-percha;
for dispensing a rinsing agent or disinfecting agent into a dental root with the additional use of a root canal probe; alternatively, a medical or dental treatment device may be used for this application, comprising a sound source, in particular an ultrasound source, a delivery device for dispensing a medium and a root canal probe;

wherein for this latter application, the root canal probe is preferably designed as a hollow probe connectable to the delivery device, and especially preferably vibrations created by the sound source, in particular the ultrasound source, are transmitted directly or indirectly to the rinsing agent or disinfecting agent, in particular via the root canal probe, to activate the rinsing agent or disinfecting agent;
for expanding a dental root canal; alternatively, a medical or dental treatment device, comprising a sound source, in particular an ultrasound source, may be used for this application;

wherein for this latter application the treatment device especially preferably has a dental file, in particular a diamond-tipped dental file;
for intraosseous dispensing of an anesthetic, in particular into a jawbone, with additional use of an intraosseous needle; alternatively, a medical or dental treatment device, comprising a sound source, in particular an ultrasound source, a delivery device for dispensing a medium and an intraosseous needle may be used for this application.

The individual applications mentioned here may be performed by means of a delivery part and/or by means of an active part.

Several of the applications mentioned above may preferably be combined to achieve a particularly time- and cost-effective treatment. During such a treatment, two or more delivery parts or at least one delivery part and at least one active part are especially preferably used together in succession. An embodiment of such a combined application is as follows: First, a delivery part having a sound source and a delivery device by means of which filling material is dispensed into a dental cavity is connected to the base part. Then, the delivery part is detached from the base part and the latter is connected to a first active part having a modeling probe, which is connected in particular to a sound source and acts on the filling material dispensed by the delivery part into the cavity by modeling and/or compressing the filling material. After compressing or modeling the filling material, the first active part is released from the base part and the base part is connected to a second active part having a radiation source in order to irradiate the filling material and cure it. Alternatively, only one active part is provided, comprising a modeling probe connected to a sound source in particular and a radiation source.

Another embodiment of a combination of several of the above-mentioned applications of the treatment device for a dental root canal treatment comprises the following steps: A borehole is created in the jawbone by transferring the vibrations of the sound source to the needle by means of a first delivery part connected to the base part and provided with an intraosseous needle and having a sound source connected to the needle. Next, an anesthetic is dispensed through the hollow intraosseous needle into the borehole and the jawbone by means of the delivery device of the first delivery part. Then, the first delivery part is detached from the base part. In a subsequent step, a first active part is connected to the base part, the first active part having a sound source and a tool connected thereto for processing the root canal, in particular a diamond-tipped file. Thus, a dental root canal that has previously been opened is expanded. In a further step, the base part is connected to a second delivery part, which is provided with a root canal probe and has a sound source and a delivery device for a rinsing agent or a disinfecting agent. With the help of the delivery device, the rinsing agent or the disinfecting agent is introduced into the root canal and is activated by means of the vibrations generated by the sound source and transferred by the root canal probe. A third delivery part which is subsequently connected to the base part has a delivery device, a sound source and optionally a heat source and serves to deliver a filling material, in particular gutta-percha, into the root canal. Then, a further active part is connected to the base part, which has a heat source and a tool to distribute, to compress or to model the gutta-percha dispensed into the root canal and/or an additional active part comprising a tool, for example, an obturation needle and a sound source, is connected to the base part, so that the vibrations transferred to the obturation needle distribute, compress or model the gutta-percha, or an active part which comprises a heat source and a tool and a sound source connected to the tool is connected to the base part in order to distribute, compress or model the gutta-percha. It is of course also possible to design a delivery part so that it can dispense different media so that for example, in the application described above instead of three delivery parts only two or even only one delivery part is needed.

The invention is explained in greater detail below on the basis of preferred embodiments and with reference to the accompanying drawings:

### BRIEF DESCRIPTION OF THE DRAWINGS

FIG. 1 shows an outside view of an embodiment of a medical or dental wireless treatment device for dispensing a medium, having a base part and a delivery part.

FIG. 2 shows a sectional diagram through the base part of the treatment device from FIG. 1.

FIG. 3 shows an embodiment of a base part with an interface for connection of a delivery part or an active part.

FIG. 4 shows an embodiment of a delivery part with an interface for connection to the base part.

FIG. 5 shows an outside view of an embodiment of a delivery part.

FIG. 6-9 show sectional diagrams through four different embodiments of delivery parts having media containers connected thereto.

FIG. 10 shows an outside view of an embodiment of a medical, in particular dental treatment device for dispensing a medium having a base part and an active part.

FIG. 11 shows a sectional diagram through the active part of the treatment device from FIG. 10.

FIG. 12 shows an outside view of an alternative embodiment of a medical, in particular dental treatment device for dispensing a medium having a base part and an active part.

FIG. 13 shows a sectional diagram through the active part of the treatment device of FIG. 12.

FIGS. 14-16 shows sectional diagrams through three different embodiments of active parts.

### DETAILED DESCRIPTION

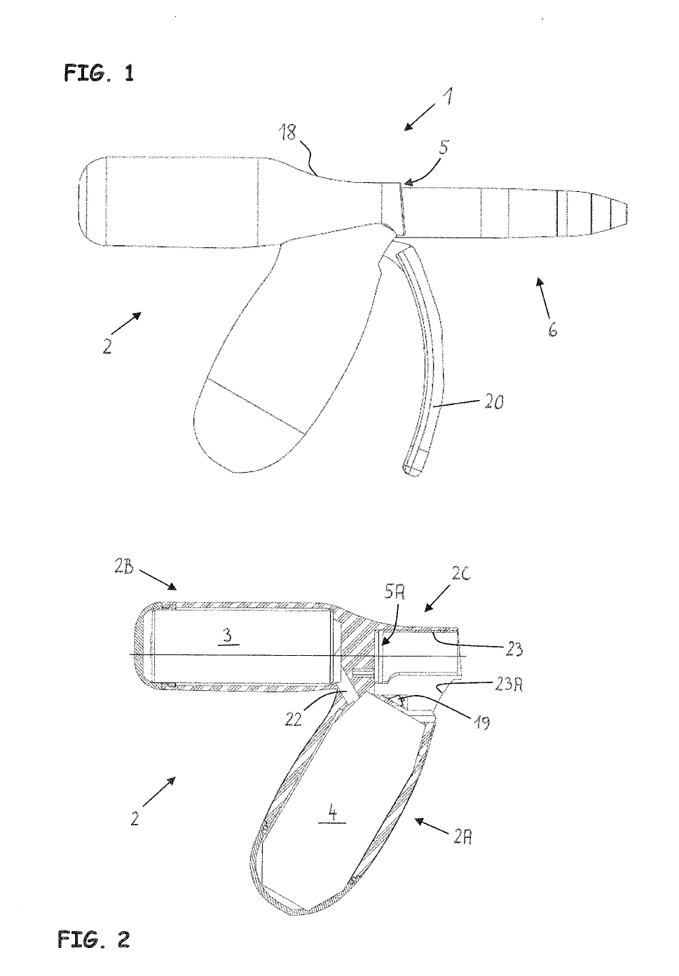

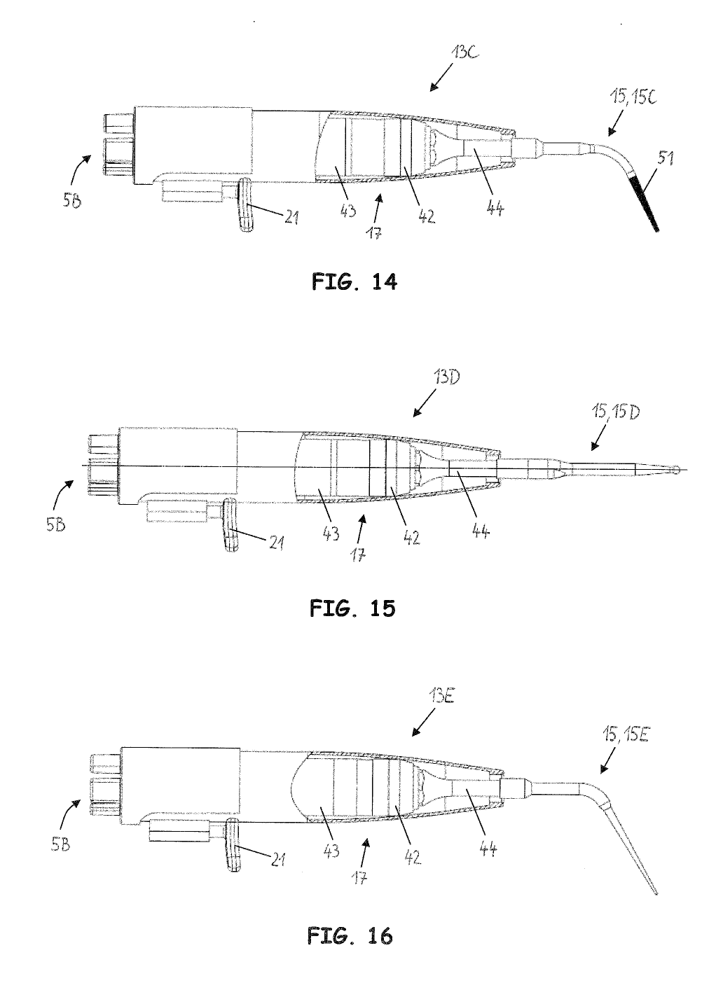

The treatment device 1 shown in FIGS. 1-16 is designed as a wireless, pistol-shaped handpiece having an outer sleeve 18, a base part 2 and multiple delivery parts 6-6E and active parts 13A-13E which can be connected to the base part 2. The connection between the base part 2 and the delivery parts 6-6E or the active parts 13A-13E is accomplished via a coupling device 5. The coupling device 5 comprises coupling elements 5A, 5B that are provided on the base part 2 and on the delivery parts 6-6E or on the active parts 13A-13E and can be interconnected.

The base part 2 comprises a handle section 2A and an extension 2B, which is arranged thereon at an angle, wherein the two are connected to one another by means of a central section 2C. The handle section 2A is designed to be hollow and holds an electrical or electronic control device 4 in its interior, comprising in particular at least one electrical or electronic component, for example, a circuit, a microcomputer, a microcontroller, a circuit board or a memory element. The control device 4 serves to control and/or regulate the treatment device 1 and the operating parameters and/or for storage of operating data or treatment data, operating or treatment sequences or cleaning data. According to one embodiment, the control device 4 is designed to recognize delivery parts 6-6E or active parts 13A-13E that can be connected to the base part and in particular to select at least one specific operating parameter for the respective connected delivery part 6-6E or active parts 13A-13E and to operate the connected delivery part 6-6E or active parts 13A-13E according to this operating parameter.

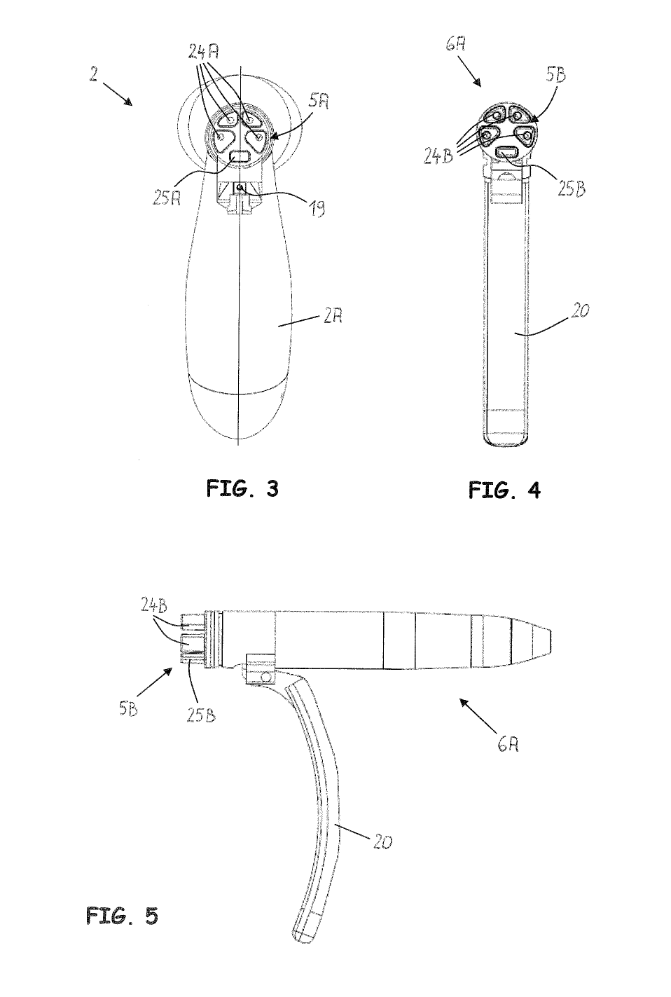

According to another embodiment, the control device 4 is designed to cooperate with one or more adjusting means and/or display devices provided on the treatment device 1 and to accomplish a unidirectional or bidirectional signal exchange or data exchange between the control device 4 and the adjusting means and/or the display devices. The control device is therefore connected, for example, to a switching element 19 (FIGS. 2, 3), which is switched via a mechanical or electrical adjusting element, for example, a lever 20, a trigger 21 or a button. For example, the treatment device 1 is activated or deactivated by operation of the adjusting element.

The protrusion 2B of the base part 2 is also designed to be hollow and holds a power supply unit 3, for example, a battery or an accumulator. To gain access to the power supply unit 3, the protrusion 2B has a cover which is detachable from the remaining protrusion.

The power supply unit 3 of the protrusion 2B and the control device 4 of the handle section 2A are connected to one another via electrical lines, wherein the lines run in a line duct 22, which connects the interiors of the protrusion 2B and the handle section 2A.

The central section 2C of the base part 2 has a receptacle 23, which can hold at least a part of the delivery part 6-6E and/or of an active part 13A-13E, in particular that part of delivery part 6-6E and/or active part 13A-13E on which the coupling element 5B is provided. The receptacle 23 is additionally preferably designed so that a part of the lever 20 or of the trigger 21 or a component operatively connected thereto can also be held therein to operate the switch element 19 arranged in the base part 2. The receptacle 23 comprises a receptacle opening 23A for a delivery part 6-6E and/or an active part 13A-13E and has at least one wall on which the coupling element 5A of the base part 2 is arranged. The coupling element 5A in particular is provided on the wall opposite the receptacle opening 23.

The coupling element 5A comprises multiple electrical contacts 24A, for example, four electrical contacts, which are connected to the power supply unit 3. Two of these contacts 24A serve to supply power to an electrical consumer of a delivery part 6-6E or of an active part 13A-13E, for example, an electrically operated sound source or ultrasound source 7, 17, of an electrically operated heat source 16 or an electrically operated light source or radiation source 14. Accordingly, multiple, in particular four, electrical mating contacts 24B are provided on each coupling element 5B of a delivery part 6-6E or an active part 13A-13E, such that when the coupling elements 5A, 5B are coupled to one another, the contacts 24A make contact and close the circuit between the power supply unit 3 and the electrical consumers by means of electrical lines. The coupling device 5 is preferably designed as a plug coupling having protrusions or pins on one coupling element 5A, 5B and receptacles or bushings for the protrusions on the other coupling element 5A, 5B, wherein the electrical contacts 24A, 24B especially preferably form the protrusions, pins, receptacles or bushings or are arranged therein. However, the coupling device 5 may of course also include other known couplings, for example, bayonet couplings or screw couplings.

In addition, an anti-rotation device, which prevents rotation of a delivery part 6-6E or an active part 13A-13E connected to the base part 2 relative to the base part 2 is provided on the coupling device 5. The anti-rotation device is preferably designed as a plug device, which in particular has a protrusion 25B and a setback 25A to receive the protrusion 25B. The protrusion 25B and the setback 25A are each provided on one of the two coupling elements 5A, 5B.

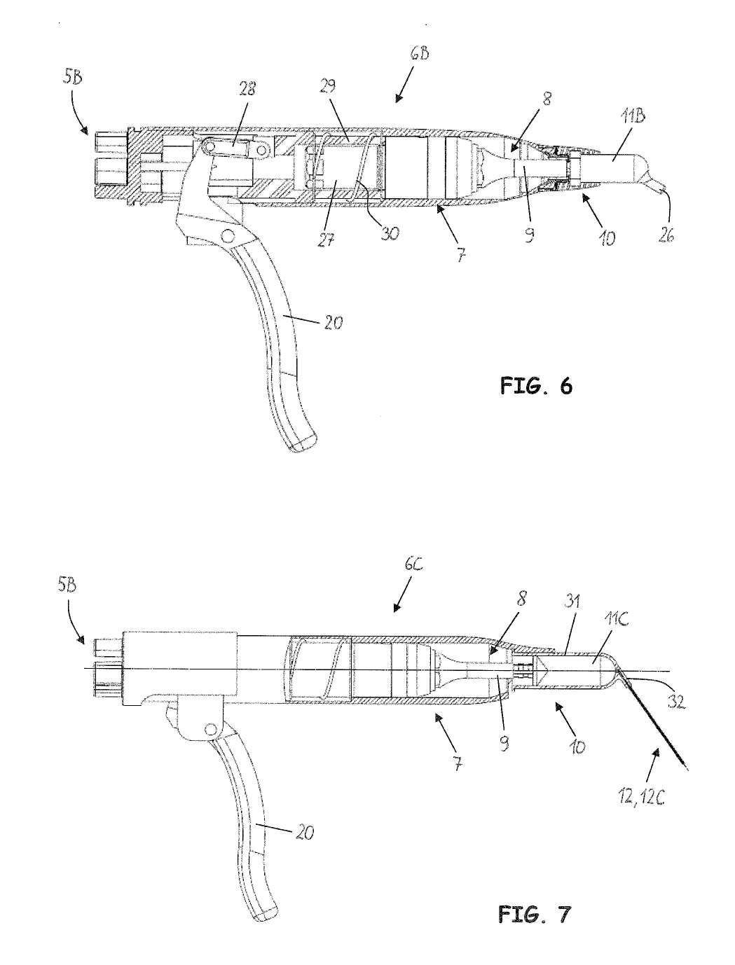

The delivery parts 6B-6E shown in FIGS. 6-9 each comprise a sound source or an ultrasound source 7, which is designed in particular as a piezoelectric, magnetostrictive or pneumatic sound source 7, a delivery device 8 for dispensing a medium from the delivery parts 6B-6E, a receptacle for the medium or a connection 10 for a media container 11B-11E and a sonotrode 9 for transferring the vibrations generated by the sound source 7 to the medium, the receptacle and/or the connection 10.

Depending on the application provided, the delivery parts 6B-6E have different features, or certain components of the various delivery parts 6B-6E have specific designs:

The delivery part 6B shown in FIG. 6 is used to dispense filling material into a dental cavity, in particular filling material, in which a change in viscosity is achieved by subjecting it to sound or ultrasound and/or heat; this filling material may be, for example, a dental cement or a filling compound based on a synthetic resin, preferably a filling compound based on a synthetic resin, which can be polymerized or cured by light, in particular a highly viscous dental filling compound based on a synthetic resin and having a high inorganic filler content. The change in viscosity is achieved by the transfer of vibrations generated by the sound source 7 to the filling material via the sonotrode 9.

The filling material is contained in a media container 11B, which is inserted into the receptacle 10 of the delivery part 6B. The vibrations generated by the sound source 7 are preferably also transferred directly or indirectly, for example, via the filler material, to the media container 11B, in particular to its dispensing nozzle 26. The receptacle 10 is preferably detachable from the delivery part 6B.

The sound source 7 comprises multiple piezoelectric elements, which are connected at one end to the sonotrode 9 and at another end to a vibrating mass or counterweight 27. The counterweight 27 is surrounded by a sliding sleeve 29.

The delivery device 8 comprises the sonotrode 9, which is arranged displaceably in the delivery part 6B, and the operating element, designed in particular as a lever 20, by means of which the sonotrode 9 can be displaced within the delivery part 6B in relation to the receptacle 10. The lever 20 is pivotably attached to the delivery part 6B and has a tappet 28 on its end arranged in the interior of the delivery part 6B, which, when the lever 20 is operated, displaces the sliding sleeve 29 and the sound source 7 including the counterweight 27 and the sonotrode 9 in the direction of the receptacle 10. Therefore, the sonotrode 9 puts the filler material under pressure or displaces it out of the delivery part 6B. In particular at its free front end, the sonotrode 9 penetrates into the receptacle 10 and/or contacts and displaces a movable plug of the media container 11B, so that the filler material is conveyed out of the delivery part 6B. The conveyance of the filling material is preferably accomplished simultaneously with the application of vibrations from the sound source 7 to the filling material via the sonotrode 9. A spring 30 serves to restore the sliding sleeve 29 and the sound source 7 including the counterweight 27 and the sonotrode 9 in the direction of the coupling element 5B as soon as the user releases the lever 20.

The delivery part 6C illustrated in FIG. 7 is used to dispense a rinsing fluid or a disinfecting fluid into a root canal. The structure of the delivery device 8 and of the sound source 7 is the same as that of the delivery part 6B of FIG. 6.

The delivery part 6C has a connection 10 for a media container or an ampoule 11C, in which the rinsing fluid or disinfecting fluid is stored. The tool 12 is designed as a hollow root canal probe 12C, which can be connected to the delivery device 8, so that rinsing fluid or disinfecting fluid delivered by the delivery device 8 can be dispensed through the probe 12C into the dental root. In addition, the root canal probe 12C can be connected to the sound source 7, so that vibrations generated by the sound source 7 can be transferred via the root canal probe 12C to the rinsing agent or disinfecting agent to activate the rinsing agent or disinfecting agent and/or to distribute and/or mix it in the dental root. The vibrations are transferred from the sonotrode 9 to the root canal probe 12C, for example, via the connection 10 and/or the ampoule 11C, in particular via the ampoule wall 31 and/or the connecting piece 32 between the ampoule and the root canal probe 12C and/or via the rinsing fluid or disinfecting fluid. The vibrations generated by the sound source 7 are also transferred directly to the rinsing agent or disinfecting agent via the sonotrode 9.

According to an alternative embodiment, the delivery part 6C does not have an ampoule for the rinsing fluid or disinfecting fluid but instead is provided with a supply line, which conducts the rinsing fluid or disinfecting fluid to or through the delivery part 6C from a rinsing fluid source or a disinfecting fluid source situated outside of the delivery part 6C. According to this alternative embodiment, the delivery part 6C also includes a sound source 7, which is connected to the root canal probe 12C, in particular via the supply line, to transfer vibrations to the root canal probe 12C and the rinsing fluid or disinfecting fluid.

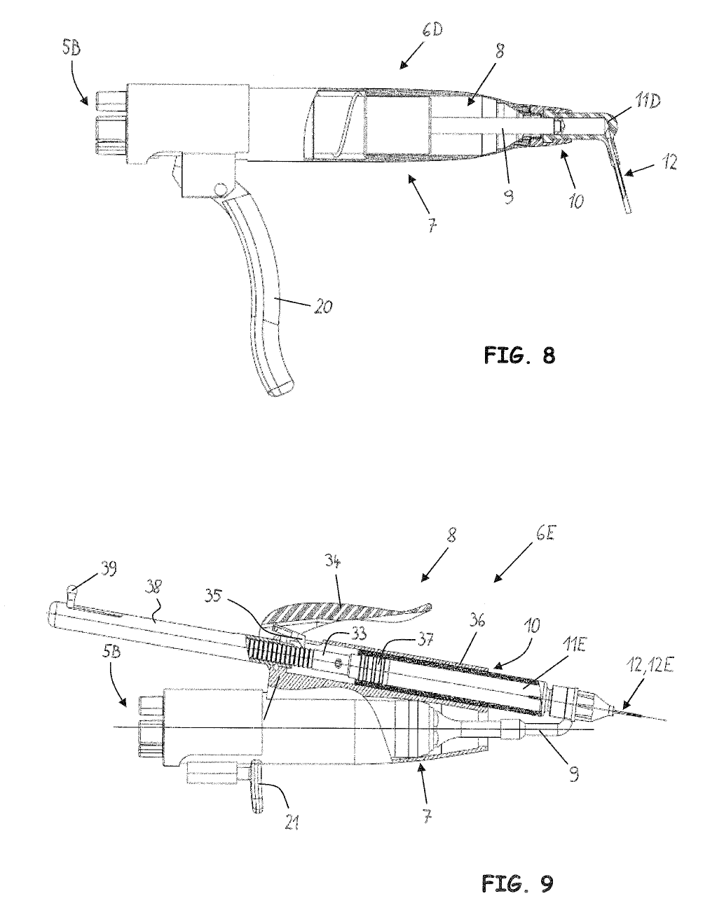

The delivery part 6D illustrated in FIG. 8 serves to dispense filling material into a dental root, in particular a filling material in which a change in viscosity is achieved by acting upon it with sound or ultrasound and/or heat; this filling material may be for example a dental cement or gutta-percha. The structure of the delivery device 8 and the sound source 7 is similar to that of the delivery part 6B in FIG. 6, wherein both the transfer of vibrations and the delivery of the filling material in particular are accomplished by the displaceable sonotrode 9.

The media container 11D for the filling material is designed as a cartridge, which has a tool 12 in the form of an elongated tube, in particular a tube that is bendable or plastically displaceable, by means of which the filling material is dispensed into the dental root. The sonotrode 9 transfers the vibrations of the sound source 7 to the filling material and also to the media container 11D, in particular to the tube 12, preferably directly or indirectly via the filling material, for example.

Alternatively or in addition to the sound source 7 and the sonotrode 9, the delivery part 6D for dispensing filling material into a dental root may also have a heat source and a heat conductor. The heat conductor transfers the heat generated by the heat source to the filling material, so that its viscosity is reduced and it can be conveyed better through the delivery device 8. The heat source preferably comprises an electrical heating element.

The delivery part 6E illustrated in FIG. 9 is designed as an intraosseous handpiece, with which a borehole can be produced in a bone, in particular in a jawbone, to allow the injection of an anesthetic. The delivery part 6E has an outer sleeve 36, a sound source 7, in particular an ultrasound source, and a sonotrode 9, wherein the sonotrode 9 is not displaceable. The sound source 7 can be turned on and off by means of the trigger 21 or a pushbutton. The delivery part 6E can be connected to a tool 12 in the form of an intraosseous needle 12E, wherein the sonotrode 9 is designed and arranged on the delivery part 6E so that it transfers the vibrations generated by the sound source 7 onto the needle 12E. The sonotrode 9 may come in direct contact with the cannula of the needle 12E, for example, or may transfer the vibrations to a cannula apron or to a receptacle of the delivery part 6E, in which the needle 12E is accommodated. The cannula of the needle 12E provided with a tip and in particular with one or more cutting edges may thus be induced to vibrate, so that it penetrates into a bone, in particular a jawbone, when placed thereon, and creates a borehole.

In addition, the delivery part 6E has an elongated receptacle 10 for a media container in the form of ampoule 11E with the anesthetic. The delivery device 8 which is connected to the receptacle 10 comprises a displaceable rack 33 and a lever 34 with a pawl 35, which engages at its free end in the row of teeth of the rack 33. The rack 33 is surrounded by a protective sleeve 38 having a slot at one end, through which a handle piece 39 connected to the rack 33 protrudes. A filling level indicator for the filling level of the ampoule 11E in the form of a scale, for example, is preferably provided on the protective sleeve 38.

The lever 34 is rotatably or pivotably attached to the delivery part 6E. By moving the lever 34 in the direction of the receptacle 10, the pawl 35 pushes the rack 33 in the direction of the ampoule 11E. When the front end of the rack 33 comes in contact with a plug 37 of the ampoule 11E, it displaces the plug 37 in the direction of the needle 12E, so that the anesthetic is forced out of the ampoule 11E. If the lever 34 is moved away from the receptacle 10, the pawl 35 is also pulled backward, away from the needle 12E, so that it engages on another tooth of the rack 33.

The lever 34 and the trigger 21 can be operated independently of one another, so that either only vibrations are transferred to the intraosseous needle 12E with the delivery part 6E or only anesthetic is delivered or anesthetic is delivered and dispensed and vibrations are transferred to the intraosseous needle 12E simultaneously.

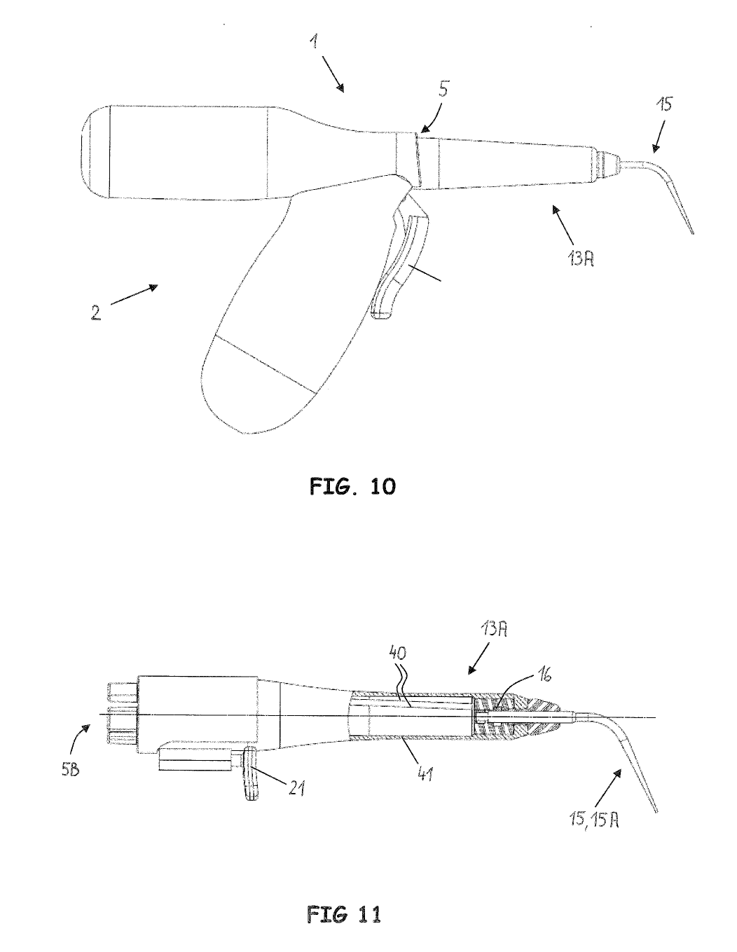

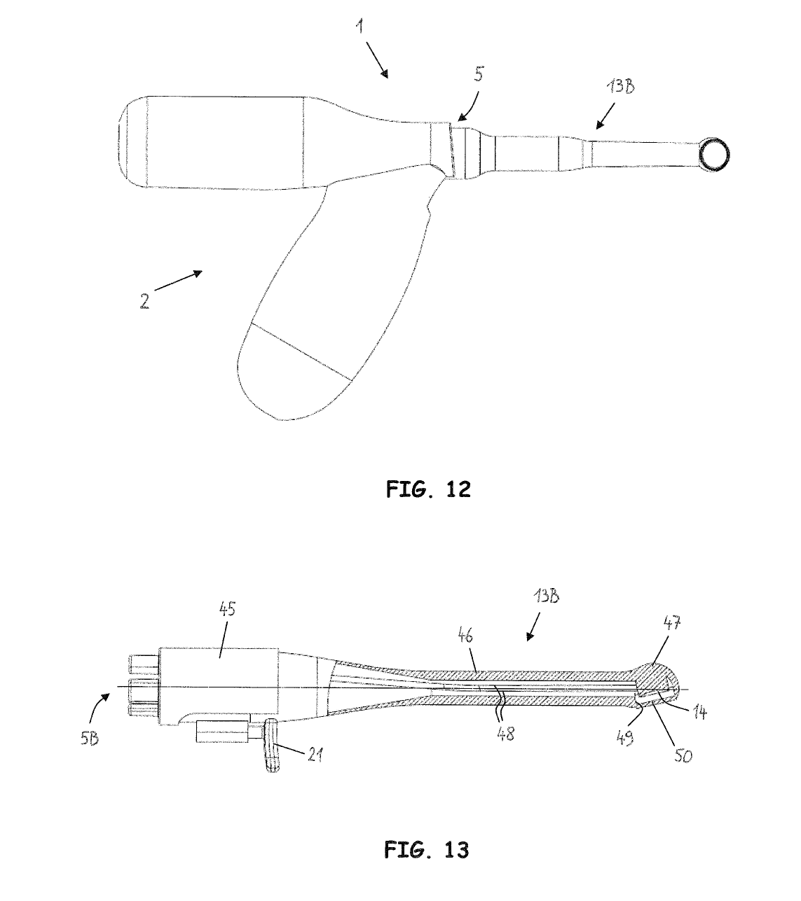

FIGS. 10-16 show different embodiments of active parts 13A-13E. All these active parts 13A-13E can be connected via the coupling device 5, 5A, 5B to the base part 2 from FIG. 2; this is illustrated as an example in FIGS. 10 and 12 for the active parts 13A and 13B. Depending on the intended application, the active parts 13A-13E have different features or certain components of the active parts 13A-13E have specific designs. In addition, the active parts 13A-13E can be subdivided according to their application into a first group of active parts for acting on the medium dispensed by a delivery part 6-6E and a second group of active parts for acting on the treatment site, to which a delivery part 6-6E has dispensed or will dispense a medium. The first group comprises, for example, the active parts 13A, 13B, 13D, 13E, and the second group comprises the active part 13C.

The active part 13A shown in FIGS. 10 and 11 is used for obturation of root canals and for shaping, modeling or compressing filling material dispensed into the root canal. It has a tool 15 in the form of an obturation needle 15A, which is detachably or undetachably connected to the active part 13A. Two electrical lines 40 supply electrical current to a heat source 16, preferably arranged in the interior of the outer sleeve 41. The heat source 16, comprising in particular an electrical heating element, is connected to the tool 15 or is designed as part of the tool 15, so that the tool 15 can be heated by the heat source 16. The tool 15 transfers heat to the filling material to reduce the viscosity thereof. The heat source 16 can be turned on and off by means of a pushbutton or trigger 21 and preferably also the temperature of the obturation needle 15A can be preset through the pushbutton or trigger 21.

The active part 13E shown in FIG. 16 also serves to obdurate root canals and to shape, model or compress the filling material dispensed into the root canal. It has a sound source 17, in particular an ultrasound source. According to one embodiment, the sound source 17 has the same design as the sound source 7 of the delivery part 6-6E and comprises a piezoelectric, magnetostrictive or pneumatic sound transducer 42, a counterweight 43 and a sonotrode 44. The sonotrode 44 is detachably or undetachably connected to the tool 15, in particular an obturation needle 15E, so that the vibrations generated by the sound source 17 can be transferred to the tool 15 and to the filling material. Due to the application of vibrations on the filling material according to a preferred embodiment, its viscosity is reduced. The sound source 17 can be turned on and off by means of a pushbutton or the trigger 21, wherein preferably also at least one operating parameter of the sound source 17, for example, the frequency or the power, can be preset through the pushbutton or trigger 21.

It is of course also possible to create an active part for obturation of root canals and for shaping, modeling or compressing filling material dispensed into the root canal which is a combination of the two active parts 13A and 13E and has a sound source 17 and a heat source 16.

The active part 13B shown in FIGS. 12 and 13 is used for dispensing electromagnetic radiation, in particular for dispensing radiation onto a filling compound based on a synthetic resin, which can be polymerized or cured by the action of light, in particular a highly viscous dental filling compound based on synthetic resin and having a high inorganic filler content.

The active part 13B has a connecting part 45, an elongated neck part 46 and a head part 47 connected to it. A radiation source 14 is provided in the interior of the active part 13B, in particular in the head part 47. The radiation source 14 preferably has one or more optical semiconductor elements or light-emitting diodes (LEDs). Two electrical lines 48 supply electrical power to the radiation source 14. The head part 47 has a radiation dispensing opening 49 through which the radiation generated by the radiation source 14 is dispensed and which is preferably sealed by a transparent safety glass 50. Additional optical components, for example, lenses, mirrors, radiation conductors or reflectors for the radiation to be emitted, may be provided in the active part 13B. A pushbutton or the trigger 21 serves to turn the radiation source 14 on and off.

Alternatively, it is also possible for the radiation source to be arranged outside of the active part and for the active part thus not to have any radiation source or any electrical lines for supplying the radiation source. Instead, a beam path and/or a radiation conductor is provided in this active part, extending from the coupling element 5B where the radiation is input to the radiation dispensing opening in the head part, and thus conducting the radiation through the active part.

The active parts 13C and 13D have the same design as the active part 13E in FIG. 16, each again having a sound source 17, in particular an ultrasound source and a pushbutton or trigger 21. The tools 15 of the two active parts 13C, 13D can be or are connected to the sound source 17, so that vibrations can be transferred to them.

The active part 13D serves to shape, model or compress the filling material that is dispensed into a cavity, in particular a dental cavity. It is detachably or undetachably connected to a tool 15 designed as a modeling probe 15D. The vibrations generated by the sound source 17 are transferred to the filling material via the tool 15, thereby lowering its viscosity.

The endodontic tool 15 in the form of an endodontic needle or dental file 15C connected detachably or undetachably to the active part 13C serves to widen a dental root canal. The dental file 15C therefore has an abrasive section 51, which is induced to vibrate by the sound source 17 and which removes tissue from the dental root canal because of its vibrational movement.

The delivery parts 6-6E and/or the active parts 13A-13E, in particular their outer sleeves, are manufactured from materials which resist negative influences such as corrosion due to sterilization or thermal disinfection.

The invention is not limited to the embodiments described here but instead comprises all embodiments and applications which apply or comprise the basic function principle of the invention. In addition, all features of all embodiments described and illustrated here can be combined with one another.

Thus, in particular different applications can be implemented by a single delivery part 6-6E or active part 13A-13E if they can be connected to different receptacles 10, media containers 11 and/or tools 15. For example, it is conceivable for media containers with different media, for example, with a filling material for a cavity and with a filling material for a root canal to be connectable to a single delivery part, so that this delivery part can be used for dispensing different media and/or for dispensing one or different media to different treatment sites. The same thing is true of the active parts, so that, for example, a single active part comprising a sound source can be connected to a file 15C, a modeling probe 15D or an obturation needle 15E.

It should also be pointed out that according to one embodiment, the treatment device 1 comprises a base part 2 and all the delivery parts 6-6E or active parts 13A-13E shown in FIGS. 1-16. According to another embodiment, the treatment device 1 does not include all these delivery parts 6-6E or active parts 13A-13E but instead includes fewer delivery parts 6-6E and/or active parts 13A-13E, in particular at least one delivery part 6-6E and one additional delivery part 6-6E or one active part 13A-13E.

The invention is not limited to the embodiments described here but instead comprises all embodiments, which employ or include the basic appropriate function principle of the invention. In addition, all features of all the embodiments described and illustrated here may be combined with one another.

What is claimed is:

1. A medical or dental treatment device for dispensing a medium, comprising
a base part with a power supply unit and an electrical or electronic control device, and
at least one delivery part detachably connectible to the base part via a coupling device and which comprises a sound source or an ultrasound source and a delivery device for dispensing the medium from the delivery part.

2. The medical or dental treatment device according to claim 1, wherein
the at least one delivery part comprises a receptacle for the medium or a connection for a media container and a sonotrode for transferring the vibrations generated by the sound source or the ultrasound source to the medium and the receptacle or the connection.

3. The medical or dental treatment device according to claim 1, wherein
the at least one delivery part comprises a radiation source for emitting a radiation which activates the medium.

4. The medical or dental treatment device according to claim 1, wherein
the at least one delivery part comprises a tool for acting on and/or delivering the medium and a sonotrode for transferring the vibrations generated by the sound source or the ultrasound source to the tool.

5. The medical or dental treatment device according to claim 1, wherein
the at least one delivery part has a heat source for heating the medium.

6. The medical or dental treatment device according to claim 1, comprising
at least one active part, which is detachably connected to the base part via the coupling device and which is configured to act on the medium dispensed by the delivery part.

7. The medical or dental treatment device according to claim 6, wherein
the at least one active part has a radiation source for emitting a radiation that activates the medium.

8. The medical or dental treatment device according to claim 6, wherein
the at least one active part comprises a tool for acting on or activating the dispensed medium.

9. The medical or dental treatment device according to claim 8, wherein
the at least one active part comprises a sound source or an ultrasound source for transferring vibrations to the tool.

10. The medical or dental treatment device according to claim 6, wherein
the at least one active part comprises a heat source for heating the medium.

11. The medical or dental treatment device according to claim 1, comprising
at least one active part detachably connectable to the base part via the coupling device and which is configured to act on the treatment site to which the delivery part has dispensed or will dispense the medium.

12. The medical or dental treatment device according to claim 11, wherein
the at least one active part comprises a tool for preparing the root canal and a sound source or an ultrasound source for transferring vibrations to the tool.

13. The medical or dental treatment device according to claim 1, wherein
the base part has pistol-shaped handle section in which at least a part of the power supply unit and/or the electrical or electronic control device is held.

14. The medical or dental treatment device according to claim 1, wherein
the treatment device is configured as a wireless handpiece, and the power supply unit is configured as a battery or an accumulator.

15. A method for dispensing filling material into a cavity, comprising
providing a medical or dental treatment device having a base part with a power supply unit and an electrical or electronic control device;
providing at least one delivery part that is detachably connected to the base part via a coupling device and comprises a sound source or an ultrasound source and a delivery device for dispensing the filling material from the delivery part; and
actuating a control on the device to selectively provide power to the sound source or the ultrasound source and cause the filling material to be dispensed.

16. The method of claim 15, wherein
the medical or dental treatment device is used for exerting an action on the filling material dispensed by the delivery part.

17. The method of claim 15, wherein
the filling material can change in viscosity when subjected to at least one of sound, ultrasound or heat.

18. A medical or dental treatment device, comprising
a base part with a power supply unit and an electrical or electronic control device,
at least one delivery part detachably connectible to the base part via a coupling device and comprising a sound source or an ultrasound source, and
a delivery device for dispensing a medium from the delivery part and a root canal probe for dispensing a rinsing agent or disinfecting agent into a dental root.

19. The medical or dental treatment device according to claim 18, wherein
the root canal probe is designed as a hollow probe connectable to the delivery device.

20. The medical or dental treatment device according to claim 18, wherein
vibrations generated by the sound source or the ultrasound source are transferred directly or indirectly via the root canal probe to the rinsing agent or disinfecting agent to activate the rinsing agent or disinfecting agent.
